SEQUENCE LISTING (5 sequences)

SEQ ID NO: 1 - protein, 8 aa, Artificial Sequence
  synthesized
NAPVSIPQ

SEQ ID NO: 2 - protein, 9 aa, Artificial Sequence
  synthesized
SALLRSIPA

SEQ ID NO: 3 - protein, 8 aa, Artificial Sequence
  synthesized
NATLSIHQ

SEQ ID NO: 4 - protein, 8 aa, Artificial Sequence
  synthesized
TAPVPMPD

SEQ ID NO: 5 - protein, 4 aa, Artificial Sequence
  Synthesized
SKIP

(19) United States
(12) Patent Application Publication
(10) Pub. No.: US 2022/0340955 A1
(43) Pub. Date: Oct. 27, 2022

(54) METHODS FOR DIAGNOSING AND TREATMENT MONITORING OF ADNP-DEFICIENT PATIENTS

(71) Applicant: RAMOT AT TEL-AVIV UNIVERSITY LTD., Tel Aviv (IL)

(72) Inventors: Illana GOZES, Tel Aviv (IL); Oxana KAPITANSKY, Tel Aviv (IL)

(21) Appl. No.: 17/760,868

(22) PCT Filed: Sep. 16, 2020
(86) PCT No.: PCT/IL2020/051010

§ 371 Date: Mar. 16, 2022

Related U.S. Application Data
(60) Provisional application No. 62/902,420, filed on Sep. 19, 2019.

(51) Int. Cl. C12Q 1/689

(52) U.S. Cl. CPC C12Q 1/689

(57) ABSTRACT

The present invention provides methods of diagnosing ADNP-deficient subjects and monitoring a treatment of such patients, as well as kits allowing the diagnosis and the monitoring.

### FIELD OF THE INVENTION

The present invention relates to methods of diagnosing and monitoring of treatment of a subject having impaired including, but not limited to mutated activity-dependent neuroprotective protein (ADNP) or a deficiency thereof.

### BACKGROUND OF THE INVENTION

Autism spectrum disorder (ASD) constitutes a complex neurodevelopmental disorder that encompasses a wide array of symptoms ranging from sensory sensitivity, social anxiety, communication difficulties, to repetitive behaviors. In addition, children diagnosed with ASD display various medical comorbidities of high prevalence. Previous research strongly implicates a role for metabolites derived from the gut microbiota as modulators of autistic behavioral phenotypes, influencing the host metabolome and shaping disease outcomes, at the individual level (Mussap et al., 2016, Expert review of molecular diagnostics 16:869-881 doi:10.1080/14737159.2016.12027652016).

The prevalence of ASD has been increasing over the past 2 decades at an alarming rate. According to a recent review of world medical records, 1 in 68 children aged 8 years was identified as having ASD. Despite the obvious prevalence, little is known about the underlying molecular mechanisms of immunopathogenesis. Neuroimaging and postmortem studies have provided evidence for disruptions in functional and structural connectivity in the brains of affected individuals. Autism presents a great challenge to science; currently, large genes that are crucial for brain development serve as key candidates for research on autism pathophysiology. In this respect, while searching for genes that shape our brains, we discovered the activity-dependent neuroprotective protein (ADNP), a highly conserved, vertebrate-specific protein, essential for brain formation and function. ADNP is a ˜124 kDa protein (coded by the human ADNP gene on chromosome 20q12-13.2) (Bassan et al. 1999, Journal of neurochemistry 72:1283-1293; Gozes et al. 2015, JAD 45:57-73 doi:10.3233/JAD-142490) that is necessary for brain development, brain plasticity, cognitive and social functioning, all of which may be impaired in ASD.

Heterozygous dominant de novo mutations in ADNP have been identified in cohorts of intellectually disabled (ID) children suffering from syndromic ASD and calculated to affect 0.17% of the autistic children—defining the ADNP syndrome (Gozes et al. 2015 Journal of molecular neuroscience: MN 56:751-757 doi:10.1007/s12031-015-0586-6; Gozes et al. 2017, Frontiers in endocrinology 8:107 doi:10.3389/fendo.2017.00107). Importantly, ADNP is one of a group of de novo mutated genes including CHD8, TBR1, SYNGAP1, and SHANK3 that lead to autism in a substantial proportion of cases with some similar fundamental mechanisms affecting synaptic function and phenotypic characteristics.

Upon discovery of ADNP, we also identified an eight amino acid peptide fragment, namely NAP (NAPSVIPQ) (Bassan et al. 1999). This peptide fortifies the interaction of ADNP with microtubule (MTs), enhances MT dynamics and autophagy, protects against electrical blockade and shows cognitive protection in preclinical and clinical studies (Bassan et al. 1999; Gozes et al. 2017, Gozes I, Editor, Neuroprotection in Alzheimer's Disease, 1st Edition, Academic Press/Elsevier Chapter 13—Pages 253-270).

Our recent results revealed that Adnp^{+/-} mice have developmental delays, impaired vocalizations, and motor dysfunction along with memory and social impairments, mimicking the ADNP syndrome in children. Exogenous administration of NAP was shown to at least partially reverse behavioral and developmental defects (Hacohen-Kleiman et al. 2018, The Journal of clinical investigation 128:4956-4969 doi:10.1172/JCI98199). In a different experimental model (i.e., gut inflammation), anti-inflammatory NAP effects were observed in human microbiota-harboring mice suffering from subacute ileitis (Escher et al., Journal of microbiology & immunology 8:34-40 doi:10.1556/1886.2018.00Do006).

WO2017130190 describes formulation for effective delivery of pharmaceutically active peptides possessing neuroprotective activity such as NAP and SKIP, and their use in treating several clinical implications such as anxiety, autism, schizophrenia etc.

Currently, the ADNP syndrome is determined through a genetic testing. While as described above administration of NAP peptide may be potentially alleviate the symptoms, no approved treatment is available. There is an ongoing need for finding treatment for ASD and for means to monitor the efficacy of treatment.

### SUMMARY OF THE INVENTION

The present invention is based on the unexpected observation that there is a statistically significant correlation between a microbiome, in particular gut microbiome, and deficiency to ADNP activity that may be caused by mutation harming ADNP activity or ADNP levels. It was also unexpectedly shown that there is a statistical correlation between the treatment of ADNP deficiently by NAP peptide and change in the microbiota levels toward those observed in healthy subjects.

According to one aspect, the present invention provides a method for monitoring a response to a treatment of a disease or condition related to deficiency of activity-dependent neuroprotective protein (ADNP) in an ADNP-deficient subject comprising measuring the amount of at least one type of bacteria of a microbiome in a biological sample obtained from the subject at two or more different time points, wherein the patient responds favorably to the treatment if the amount of said bacteria in the later measurement changes in amount to a value which is closer to the average amount of said bacteria in subjects having normal levels of active ADNP than in the previous measurement. According to some embodiment, the biological sample is a fecal sample and the microbiome is a gut microbiome.

Thus, according to one embodiment, the present invention provides a method for monitoring a response to a treatment of a disease or condition related to a deficiency of activity-dependent neuroprotective protein (ADNP) in an ADNP-deficient subject comprising measuring the amount of at least one type of bacteria of a gut microbiome in a biological sample obtained from the subject at two or more different time points, wherein the patient responds favorably to the treatment if the amount of said bacteria in the later measurement changes in amount to a value which is closer to the average amount of said bacteria in subjects having normal levels of active ADNP than in the previous measurement.

According to one embodiments, the subject is a male subject. According to some embodiments, the at least one type of bacteria of the gut microbiome is selected from Eubacteria, Lactobacillus group, Bifidobacterium, and Clostridium coccoides. According to certain embodiments, a decrease in the amount of the at least one type of bacteria between the previous and the later measurements is indicative of the favorable response of the subject to treatment.

According to other embodiments, the subject is a female subject. According to some embodiments the at least one type of bacteria of the gut microbiome is selected from Enterococcus genus, Lactobacillus, Clostridium coccoides and Clostridium cluster. According to one embodiments, a decrease in the amount of at least one type of bacteria selected from Enterococcus and Clostridium coccoides and/or an increase in the amount of at least one type of bacteria selected from Lactobacillus and Clostridium cluster IV between the previous and the later measurements is indicative of the favorable response of the subject to treatment.

According to some embodiments, the treatment comprises administering a pharmaceutical composition comprising an active agent selected from NAP peptide, SKIP peptide, and ketamine.

According to another aspect, the present invention provides a method of detecting a subject suffering from a deficiency of activity-dependent neuroprotective protein (ADNP), the method comprising:

(i) measuring the amount of at least one type of bacteria in the microbiome of the subject; and
(ii) comparing the amount determined in (i) to the average amount of the bacteria in subjects having normal ADNP levels,
wherein a significant difference between of the amount of said bacteria in comparison to the average amount in subjects having normal levels of active ADNP is predictive of ADNP deficiency. According to some embodiment, the microbiome is gut microbiome.

According to some embodiments, the subject is a male subject. According to some embodiments, the at least one type of bacteria of gut microbiome is bacteria selected from Eubacteria, Lactobacillus group, Bifidobacterium, and Clostridium coccoides. According to some embodiments, a significant increase in the amount of the at least one type of the bacteria in comparison to the average amount of the type of bacteria in subjects having normal ADNP levels is predictive of ADNP deficiency in the subject.

According to other embodiments, the subject is a female subject. According to some embodiments, the at least one type of bacteria of the gut microbiome is bacteria selected from Enterococcus genus, Lactobacillus, Clostridium coccoides and Clostridium cluster IV. According to other embodiments, a significant increase in the amount of bacteria selected from Enterococcus and Clostridium coccoides and/or an significant decrease in the amount of bacteria stain selected from Lactobacillus and/or Clostridium cluster IV in comparison to the average amount of the bacteria in subjects having normal ADNP levels is predictive of ADNP deficiency in the subject.

According to some embodiments, the method further comprises providing recommendations to treat a disease or condition related to ADNP-deficiency.

According to some embodiments, the method further comprises consulting to monitor a response to the treatment of the present invention.

According to any one of the embodiments and aspects of the present invention, the ADNP-deficiency related disease or condition is selected from Autism spectrum disorder (ASD), Alzheimer's disease (AD), Parkinson's disease (PD), schizophrenia, attention deficit disorder (ADHD), post-traumatic stress syndrome (PTSD), tauopathy of various causes, mild cognitive impairment, anxiety, depression, and muscle dysfunction/muscle disease.

According to any one of the embodiments and aspects of the present invention, the treatment comprises administering a pharmaceutical composition comprising an active agent selected from NAP peptide, SKIP peptide and ketamine to the subject.

According to another aspect, the present invention provides a kit for monitoring a response to a treatment of an ADNP-deficiency related disease or condition of an ADNP-deficient subject, the kit comprising: means for quantifying the amount of at least one type of bacteria in two or more samples of a microbiome of the subject at different time points and instructions to compare the amount of the at least one type of bacteria with the amount of said bacteria in a two samples from the subject, wherein the amount of said bacteria in the later measurement being closer to the average amount of said bacteria in subjects having normal levels of active ADNP is indicative of the efficiency of the treatment. According to some embodiments, the microbiome is a gut microbiome.

According to another aspect, the present invention provides a kit for determining a subject suffering from ADNP-deficiency, the kit comprises (i) means for quantifying the amount of at least one type of bacteria in a sample of a microbiome of the subject and (ii) means for comparing the amount obtained in (i) to the average amount of said at least one type of bacteria in subjects having normal levels of active ADNP, wherein a difference between the amount of (i) and the average amount of said type of bacteria in subjects having normal levels of active ADNP is indicative of ADNP-deficiency. According to some embodiments, the microbiome is a gut microbiome.

### BRIEF DESCRIPTION OF DRAWINGS

FIG. 1 shows the results of quantities Real Time PCR analysis of 5 bacterial genera and species in Adnp^{+/-} male mice, Adnp^{+/+} male mice and Adnp^{+/-} male mice treated with NAP for which significant sex-dependent differences were detected between Adnp^{+/+} and Adnp^{+/-} male mice mostly coupled with corrective effects of NAP treatment. FIG. 1A—Eubacteriaceae (EubV3); FIG. 1B—Lactobacillus group (Lacto), FIG. 1C—Bifidobacterium genus (BIF); FIG. 1D—Clostridium coccoides group (Coer), and FIG. 1E—Mouse intestinal Bacteroides (MIB).

FIG. 2 shows the results of quantities Real Time PCR analysis of 4 bacterial genera and species in Adnp^{+/-} female mice, Adnp^{+/+} female mice and Adnp^{+/-} female mice treated with NAP for which significant sex-dependent differences were detected and differences between Adnp^{+/+} and Adnp^{+/-} female mice. FIG. 2A—Enterococcus genus (gEncocc); FIG. 2B—Lactobacillus group (Lacto), FIG. 2C—Clostridium coccoides group (Coer), and FIG. 2D—Clostridium Cluster IV, sgClep.

For FIGS. 1 and 2, the bacterial groups abundant in the murine intestinal microbiota (males: Adnp^{+/+} n=15, Adnp^{+/-} n=8, Adnp^{+/-} NAP, n=11; females: Adnp^{+/+} n=6, Adnp^{+/-} n=10, Adnp^{+/-} NAP, n=10). Results were normalized to 16S DNA. Two-way ANOVA analysis with Fisher's LSD as post hoc revealed significant differences between DD treated Adnp^{+/+}, Adnp^{+/-} and NAP treated Adnp^{+/-} mice (*P<0.05, **P<0.01 and ***p<0.001).

FIG. 3. Shows behaviors of Adnp^{+/-} and Adnp^{+/-} NAP treated mice in open field test in comparison to healthy mice. FIG. 3A—frequency entrance to the center. Male mice showed significantly higher frequency entrance in to the center area compared to Adnp^{+/+} mice and NAP treated Adnp^{+/-} mice as revealed by Two-way ANOVA analysis with Fisher's LSD as post hoc (**p<0.01 and *p<0.05, respectively). Furthermore, sex differences were observed in Adnp^{+/+} group (**p<0.01). FIG. 3B—time spend in the center. Time spent in the center area did not reveal significant changes. FIG. 3C—time spent in locomotor activity. Adnp^{+/-} males showed significantly higher percentage of time spent with locomotion activity (%) compared to Adnp^{+/+} mice as revealed by Two-way ANOVA analysis with Fisher's LSD as post hoc (**p<0.01). Furthermore, sex differences were observed in Adnp^{+/+} group (*p<0.05), Adnp^{+/-} group (*p<0.05) and NAP treated Adnp^{+/-} mice group (*p<0.05). FIG. 3D.—total distance traveled by mice. Adnp^{+/-} male mice traveled longer distances in the arena compared to Adnp^{+/+} mice as revealed by Two-way ANOVA analysis with Fisher's LSD as post hoc (***p<0.001). Also, sex dependent differences were observed in the Adnp^{+/+} group (**p<0.01). FIG. 3E. Velocity of mice. Adnp^{+/-} male mice showed significant higher velocity in the arena compared to Adnp^{+/+} mice as revealed by Two-way ANOVA analysis with Fisher's LSD as post hoc (***p<0.001). Also, sex dependent differences were observed in Adnp^{+/+} group (**p<0.01).

FIG. 4 shows a tabular summary of correlation between changes in gut bacterial groups and changes in behavior. As the data did not show normal distribution, we employed the recommended Spearman's correlation comparing expression levels of each bacterial group relative to behavioral test results. Spearman correlation coefficients as well as the p values of for bacteria group showed a significant correlation to behavioral changes are presented in the table. Female results are presented in bold.

### DETAILED DESCRIPTION OF THE INVENTION

The present invention is based on the unexpected observation that the content of microbiota, and in particular gut microbiota, may be used for diagnosing an activity-dependent neuroprotective protein (ADNP) deficiency, and monitoring changes in microbiota content during the treatment may be correlated to the efficacy of the treatment of ADNP-deficient subjects. Thus, sampling microbiota may be an efficient and convenient tool for diagnosing ADNP-deficient (such as ADNP mutated or having aberrant ADNP activity) subjects and monitoring their treatment and progress.

According to one aspect, the present invention provides a method for monitoring a response to a treatment of disease or condition related to a deficiency of activity-dependent neuroprotective protein (ADNP) in an ADNP-deficient subject, the method comprises measuring the amount of at least one type of bacteria of a microbiome in a biological sample obtained from the subject at two or more different time points, wherein the patient responds favorably to the treatment if the amount of said bacteria in the later measurement changes in amount to a value which is closer to the average amount of said bacteria in subjects having normal levels of active ADNP than in the previous measurement.

The term “activity-dependent neuroprotective protein (ADNP)” refers to a human protein (factor) which sequence has the accession number NP 852107. The protein has neurotrophic/neuroprotective activity as measured e.g. with in vitro cortical neuron culture assays described by, e.g., Gozes et al., Proc. Natl. Acad. Sci. USA 93, 427-432 (1996) and reviewed in Gozes et al. Book Chapter 2017, above).

The terms “microbiota” and “microflora” are used herein interchangeably and refer to the population of all the microorganisms in or on the individual, including bacteria, archaea, yeasts, and parasites. According to some embodiments, microbiota refers to the population of all the microorganisms in the gastrointestinal tract of the individual. According to one embodiment, the microbiota refers to the population of bacteria in the gastrointestinal tract.

The term “microbiome” refers, collectively, to the collection of microbial genomes of the microbiota. According to some embodiments, the microbiome is a gut microbiome (i.e. intestinal microbiome).

Any known method for sampling microbiota and microbiome may be used.

The terms “gastrointestinal” and “GI” are used herein interchangeably and refer to the stomach and intestines in the digestive tract of humans and other animals. However, as also used in the context herein, the term “gastrointestinal tract” (“GI tract”) refers to the entire alimentary canal, from the oral cavity to the rectum. The term encompasses the tube that extends from the mouth to the anus, in which the movement of muscles and release of hormones and enzymes digest food. The gastrointestinal tract starts with the mouth and proceeds to the esophagus, stomach, small intestine, large intestine, rectum and, finally, the anus. As used herein, the terms “gastrointestinal” and “GI tract” are not intended to include accessory organs of digestion, such as the liver, gallbladder, and pancreas.

The term “commensal” refers to organisms that are normally harmless to a host, and can also establish mutualistic relations with the host.

The term “deficiency” refers to an aberrant activity of ADNP that may be a result to lower levels of the intact protein or of mutations reducing or impairing the activity of the mutated ADNP. According to one embodiment, the aberrant activity of ADNP is a reduced activity.

The term “type” refers to bacterial genera, species and strains.

As used herein, the term “genus” refers to a taxonomic rank which is above species and below family. As used herein, the term “quantitative measure” refers to presence or absence, absolute or relative amounts or concentrations, absolute or relative increases or decreases and discrete or continuous ranges (e.g., a number, a degree, a level, a threshold, a quantile or a bucket). In some embodiments, the quantitative measure can be an absolute value, a ratio, an average, a median, or a range of numbers.

The microbiome may be analyzed by any known method in the art. According to some embodiment, preforming quantity analyses of the microbiome comprises performing qPCR, WGS, mass spectroscopy, microarray analysis or DNA or RNA sequencing, loop-mediated isothermal amplification, nucleic acid sequence-based amplification, strand displacement amplification, multiple displacement amplification or other isothermal amplification methods on nucleic acids of microbes in the sample. Quantitative measures of the specified genera or species can be determined, for example, by selective amplification of 16S rRNA characteristic of each genus or species. According to some embodiments, the specified genera or species can be quantified using qPCR or sequencing methodologies. According to some embodiments, measuring the amount of at the at least one type of bacteria comprises RNA 16S analysis and quantification the microbiome. According to some embodiments, measuring the amount of at the at least one type of bacteria comprises quantitative measures of 16S RNA of the microbiome. According to some embodiments, the analysis comprises 16S rRNA sequencing and quantification.

The terms “bacterial load” and “amount of bacteria” are used herein interchangeably and refer to measurable quantity of bacteria. This measurement may include but are not limited to the population count, density, colony forming units (CFU), or other known procedures in the art such as turbidity measurements. The bacterial load is typically expressed as colony forming units (CFU) per ml, per gram of sample or tissue, or per surface area.

According to the concept of the present invention, the microbiota or microbiome of a subject is sampled in two distinct time points, the content of the microbiota or microbiome is quantified and compared between the two points. The difference in bacterial load of certain bacterial genera and/or species which is directed towards the average amount of said genera and/or species in subjects having normal levels of active ADNP corresponds to a favorable response to the treatment. In other words, the favorable response is observed if the bacterial load of the particular bacterial genera and/or species in the later sample is closer to the average bacterial load in subjects that do not suffer from ADNP deficiency than in the previous sample.

According to some embodiments, one of the samples is obtained before the initiation of the treatment. According to some embodiments, the method comprises measuring the bacterial load in 3 different samples obtained in 3 different time points. According to some embodiments, the method comprises measuring the bacterial load in 4 different samples obtained in 4 different time points. According to one embodiment, one of the said points is before initiation of the treatment.

According to one embodiment, the biological sample is saliva and sweat sample (skin microbiome). In some embodiments, the microbiota sample is a fecal sample. In other embodiments, the microbiota sample is retrieved directly from the intestine.

Any known method for sampling gut microbiome may be used, e.g. as described in Tang et al., Front. Cell. Infect. Microbiol. 10:151, 2020; doi:10.3389/fcimb.2020.00151 et al. The sample of a gut microbiome is preferably obtained by non-invasive means. According to some embodiments, the biological sample is a fecal sample.

According to one embodiment, the microbiome sample (e.g. fecal sample) is frozen and/or lyophilized prior to analysis. According to another embodiment, the sample may be subjected to solid phase extraction methods. According to some embodiments, the biological sample is selected from feces, mucosal biopsy, intestinal fluid.

According to one embodiment, the sample is a fecal sample and the microbiome is a gut microbiome. Thus, according to some embodiments, the present invention provides a method for monitoring a response to a treatment of disease or condition related to a deficiency of activity-dependent neuroprotective protein (ADNP) in an ADNP-deficient subject, the method comprises measuring the amount of at least one type of bacteria of a gut microbiome in a biological sample obtained from the subject at two or more different time points, wherein the patient responds favorably to the treatment if the amount of said bacteria in the later measurement changes in amount to a value which is closer to the average amount of said bacteria in subjects having normal levels of active ADNP than in the previous measurement.

According to some embodiment, the at least one type of bacteria of the gut microbiome is selected from Bifidobacterium, Clostridium, Bacteroides, Faecalibacterium, Eubacterium, Ruminococcus, Peptococcus, Peptostreptococcus, Parabacteroides, Escherichia coli, Verrucomicrobia and Lactobacillus. According to some embodiments, the method comprises comparing the amount of bacteria of Clostridium genera in two biological samples. According to one embodiment, the method comprises comparing the amount of bacteria of Bifidobacterium genera in two biological samples. According to another embodiment, the method comprises comparing the amount of bacteria of Bacteroides genera in two biological samples. According to a further embodiment, the method comprises comparing the amount of bacteria of Faecalibacterium genera in two biological samples. According to a certain embodiment, the method comprises comparing the amount of bacteria of Eubacterium genera in two biological samples. According to one embodiment, the method comprises comparing the amount of bacteria of Ruminococcus genera in two biological samples. According to a further embodiment, the method comprises comparing the amount of bacteria of Peptococcus genera in two biological samples. According to yet another embodiment, the method comprises comparing the amount of bacteria of Peptostreptococcus genera in two biological samples. According to a further embodiment, the method comprises comparing the amount of bacteria of Parabacteroides genera in two biological samples. According to a further embodiment, the method comprises comparing the amount of Escherichia genera in two biological samples. According to a certain embodiment, the method comprises comparing the amount of bacteria of Verrucomicrobia genera in the two biological samples. According to a further embodiment, the method comprises comparing the amount of bacteria of Lactobacillus genera in two biological samples. According to any one of the above embodiments, the method comprises a comparison of two said bacteria type, genera or species. According to some embodiments, the method comprises comparison of three said bacteria types, genera or species. According to other embodiments, the method comprises comparison of four said bacteria types, genera or species. According to yet another embodiment, the method comprises comparison of five said bacteria types, genera or species. According to any one of the above embodiments, the sample is a sample of gut microbiome. According to any one of the above embodiments, the method further comprises comparing the amount of said bacteria in said samples to an average amount of said bacteria in subjects having normal levels of active ADNP, wherein change in the amount of said bacteria from previous to later samples towards the normal ADNP levels is indicative of favorable response to the treatment.

According to any one of the above embodiments, the subject is human male subject. According to some embodiments, the subject is human male subject and the at least one type of bacteria of the gut microbiome is selected from Eubacteria, Lactobacillus group, Bifidobacterium, and Clostridium coccoides. According to one embodiment, the method comprises comparing the amount of bacteria of Eubacteria in the two biological samples. According to another embodiment, the method comprises comparing the amount of bacteria of Bifidobacterium in the two biological samples. According to yet another embodiment, the method comprises comparing the amount of bacteria of Lactobacillus group in the two biological samples. According to some embodiments, the method comprises comparing the amount of Clostridium coccoides in the two biological samples. According to any one of the above embodiment, the method comprises comparison of two said bacteria types, genera or species. According to some embodiments, the method comprises comparison of three said bacteria types, genera or species. According to other embodiments, the method comprises comparison of four said bacteria types, genera or species. According to any one of the above embodiments, the sample is a sample of gut microbiome. According to some embodiments, a decrease in the amount of the at least one type of bacteria between the previous and the later measurements is indicative of the favorable response of the subject to treatment.

Thus, according to one embodiment, the present invention provides a method for monitoring a response to treatment of an ADNP deficiency related disease or condition in an ADNP-deficient male subject comprising measuring the amount of at least one type of bacteria selected from Eubacteria, Lactobacillus group, Bifidobacterium, and Clostridium coccoides of a gut microbiome in a biological sample obtained from the subject at two or more different time points, wherein the patient responds favorably to the treatment if the amount of said bacteria in the later sample is lower than the amount of said bacteria in previous sample.

According to one embodiment, the present invention provides a method for monitoring a response to treatment of an ADNP deficiency related disease or condition in an ADNP-deficient male subject comprising measuring the amount of bacteria of Bifidobacterium genus in gut microbiome in a biological sample obtained from the subject at two or more different time points, wherein the patient responds favorably to the treatment if the amount of said bacteria in the later sample is lower than the amount of said bacteria in previous sample.

According to one embodiment, the present invention provides a method for monitoring a response to treatment of an ADNP deficiency related disease or condition in an ADNP-deficient male subject comprising measuring the amount of bacteria of Eubacteria genus in gut microbiome in a biological sample obtained from the subject at two or more different time points, wherein the patient responds favorably to the treatment if the amount of said bacteria in the later sample is lower than the amount of said bacteria in previous sample.

According to one embodiment, the present invention provides a method for monitoring a response to treatment of an ADNP deficiency related disease or condition in an ADNP-deficient male subject comprising measuring the amount of bacteria of Lactobacillus group genus in gut microbiome in a biological sample obtained from the subject at two or more different time points, wherein the patient responds favorably to the treatment if the amount of said bacteria in the later sample is lower than the amount of said bacteria in previous sample.

According to one embodiment, the present invention provides a method for monitoring a response to treatment of an ADNP deficiency related disease or condition in an ADNP-deficient male subject comprising measuring the amount of Clostridium coccoides in gut microbiome in a biological sample obtained from the subject at two or more different time points, wherein the patient responds favorably to the treatment if the amount of said bacteria in the later sample is lower than the amount of said bacteria in previous sample.

According to some embodiments, the subject is human a female subject.

According to some embodiments, female and the at least one types of bacteria of the gut microbiome is selected from Enterococcus genus, Lactobacillus, Clostridium coccoides and Clostridium cluster IV. According to one embodiment, the method comprises comparing the amount of bacteria of Enterococcus genus in the two biological samples. According to another embodiment, the method comprises comparing the amount of bacteria of Lactobacillus genus in the two biological samples. According to yet another embodiment, the method comprises comparing the amount of Clostridium coccoides in the two biological samples. According to one embodiment, the method comprises comparing the amount of bacteria of Clostridium cluster IV in the two biological samples. According to any one of the above embodiments, the method comprises comparing of two said bacteria types, genera or species. According to some embodiments, the method comprises comparing of three said bacteria types, genera or species. According to other embodiments, the method comprises comparing of four said bacteria types, genera or species. According to any one of the above embodiments, the sample is a sample of gut microbiome. According to any one of the above embodiments, the method further comprises comparing the amount of said bacteria in said samples to the average amount of said bacteria in subjects having normal levels of active ADNP, wherein change in the amount of said bacteria from previous to later samples towards the normal value is indicative of favorable response to the treatment.

According to one embodiment, the present invention provides a method for monitoring a response to treatment of an ADNP deficiency related disease or condition in an ADNP-deficient female subject comprising measuring the amount of bacteria of Enterococcus genus in gut microbiome in a biological sample obtained from the subject at two or more different time points, wherein the patient responds favorably to the treatment if the amount of said bacteria in the later sample is lower than the amount of said bacteria previous sample.

According to one embodiment, the present invention provides a method for monitoring a response to a treatment of an ADNP deficiency related disease or condition in an ADNP-deficient female subject comprising measuring the amount Clostridium coccoides in gut microbiome in a biological sample obtained from the subject at two or more different time points, wherein the patient responds favorably to the treatment if the amount of said bacteria in the later sample is lower than the amount of said bacteria in previous sample.

According to one embodiment, the present invention provides a method for monitoring a response to a treatment of an ADNP deficiency related disease or condition in an ADNP-deficient female subject comprising measuring the amount of bacteria of Lactobacillus genus in gut microbiome in a biological sample obtained from the subject at two or more different time points, wherein the patient responds favorably to the treatment if the amount of said bacteria in the later sample is higher than the amount of said bacteria in previous sample.

According to one embodiment, the present invention provides a method for monitoring a response to a treatment of an ADNP deficiency related disease or condition in an ADNP-deficient female subject comprising measuring the amount of bacteria of Clostridium cluster IV in gut microbiome in a biological sample obtained from the subject at two or more different time points, wherein the patient responds favorably to the treatment if the amount of said bacteria in the later sample is higher than the amount of said bacteria in previous sample.

According to any one of the above embodiments, the treatment of ADNP-deficiency relates to any known treatment. According to one embodiment, the treatment comprises administering a pharmaceutical composition comprising NAP peptide having amino acid sequence NAPVSIPQ (SEQ ID NO: 1). According to another embodiment, the treatment comprises administering a pharmaceutical composition comprising SAL peptide having amino acid sequence SALLRSIPA (SEQ ID NO: 2). According to yet another embodiment, the treatment comprises administering a pharmaceutical composition comprising SKIP peptide (SEQ ID NO: 5). According to some embodiments, the treatment comprises administering a pharmaceutical composition comprising an active agent selected from Acetyl-SKIP-NH2 Acetyl-NAP-NH2, NAT peptide having amino acid sequence NATLSIHQ (SEQ ID NO: 3); TAP peptide having amino acid sequence TAPVPMPD (SEQ ID NO: 4) or NAP alpha-aminoisobutyric acid (IsoNAP) or the d-amino acid derivative thereof. According to some embodiments, the treatment comprises administering a pharmaceutical composition comprising ketamine.

According to some embodiments, the ADNP-deficiency comprises reduced levels of active ADNP. According to another embodiment, the ADNP-deficiency comprises a reduced activity of ADNP due to a mutation in ADNP. According to another embodiment, the ADNP-deficiency comprises an aberrant activity of ADNP.

According to one embodiment, the ADNP-deficiency related disease or condition is selected from autism spectrum disorder (ASD), Alzheimer's disease (AD), Parkinson's disease (PD), schizophrenia and attention deficit disorder (ADHD), post-traumatic stress syndrome (PTSD), tauopathy, mild cognitive impairment, anxiety, depression, and muscle disease/dysfunction. According to one embodiment, the disease related to ADNP-deficiency is autism spectrum disorder (ASD).

According to one embodiment, the present invention provides a method for monitoring a response to a treatment of autism spectrum disorder (ASD) in a male subject, the method comprises comparing the amount of bacteria selected from Eubacteria, Lactobacillus group, Bifidobacterium, and Clostridium coccoides in a biological sample of gut microbiome of the subject at two or more different time points, wherein the patient responds favorably to the treatment if the amount of said bacteria in the later measurement changes in amount to a value which is closer to the average amount of said bacteria in subjects having normal levels of active ADNP than in the previous measurement. According to one embodiment, the method comprises measuring the amount of bacteria of Bifidobacterium genus in gut microbiome in a biological sample obtained from the subject at two or more different time points. According to one embodiment, the treatment is administering a pharmaceutical composition comprising NAP peptide.

According to one embodiment, the present invention provides a method for monitoring a response to a treatment of autism spectrum disorder (ASD) in a female subject, the method comprises comparing the amount of bacteria selected from Enterococcus genus, Lactobacillus, Clostridium coccoides and Clostridium cluster IV in a biological sample of gut microbiome of the subject at two or more different time points, wherein the patient responds favorably to the treatment if the amount of said bacteria in the later measurement changes in amount to a value which is closer to the average amount of said bacteria in subjects having normal levels of active ADNP than in the previous measurement. According to one embodiment, the method comprises measuring the amount of bacteria of Clostridium cluster IV genus in gut microbiome in a biological sample obtained from the subject at two or more different time points. According to one embodiment, the treatment is administering a pharmaceutical composition comprising NAP peptide.

According to another embodiment, the sample is a saliva sample and the microbiome is a saliva microbiome. According to some embodiments, alternation in at least one of the following types of bacteria in monitored: Porphyromonas, Solobacterium, Haemophilus, Corynebacterium, Cellulosimicrobium, Streptococcus and Campylobacter, Porphyromonas gingivalis, Tannerella forsythia, Treponema denticola, Prevotella intermedia, Aggregatibacter actinomycemtomitans, Bacteroidetes, Firmicutes, Proteobacteria, Fusobacteria, Actinobacteria, Neisseria, Veillonella and Fusobacterium. Change in the level of at least one of said bacteria in a later sample toward its to the normal value in comparison to the bacterial load in previous sample is indicative to positive response. The term “alteration” has the meaning of an increase or decrease of the parameter or its value. According to another aspect, the present invention provides a method of detecting a subject suffering from a deficiency of activity-dependent neuroprotective protein (ADNP), the method comprising:

measuring the amount of at least one type of bacteria in the microbiome of the subject; and comparing the amount determined in (i) to the amount of the bacteria in subjects having normal ADNP levels,
wherein a significant difference between of the amount of said bacteria in comparison to the average amount in subjects having normal levels of active ADNP is predictive of ADNP deficiency. Therefore, the method is a diagnostic method.

All definitions and embodiments related to previous aspects apply for this aspect as well.

According to some embodiments, measuring the amount of at the at least one type of bacteria comprises RNA 16S analysis of the microbiome. According to some embodiments, the analysis comprises 16S rRNA sequencing and quantification. According to some embodiments, the specified genera or species can be quantified using qPCR or sequencing methodologies.

According to one embodiment, the biological sample is selected from fecal, saliva and sweat sample.

According to one embodiment, the biological sample is a fecal sample. Thus, according to one embodiment, the present invention provides a method of detecting a subject suffering from a deficiency of activity-dependent neuroprotective protein (ADNP), the method comprising:
(i) measuring the amount of at least one type of bacteria in the gut microbiome of the subject; and
(ii) comparing the amount determined in (i) to the amount of the bacteria in subjects having normal ADNP levels,
   wherein a significant difference between of the amount of said bacteria in comparison to the average amount in subjects having normal levels of active ADNP is predictive of ADNP deficiency.

According to some embodiment, the at least one type of bacteria is of gut microbiome is selected from Bifidobacterium, Clostridium, Bacteroides, Faecalibacterium, Eubacterium, Ruminococcus, Peptococcus, Peptostreptococcus, Parabacteroides, Escherichia coli, Verrucomicrobia and Lactobacillus. According to some embodiments, the method comprises measuring the amount of bacteria of Clostridium genera in the biological sample. According to one embodiment, the method comprises comparing the amount of bacteria of Bifidobacterium genera in the biological sample. According to another embodiment, the method comprises measuring the amount of bacteria of Bacteroides genera in the biological sample. According to a further embodiment, the method comprises measuring the amount of bacteria of Faecalibacterium genera in the biological sample. According to a certain embodiment, the method comprises measuring the amount of bacteria of Eubacterium genera in the biological sample. According to one embodiment, the method comprises measuring the amount of bacteria of Ruminococcus genera in the biological sample. According to a further embodiment, the method comprises measuring the amount of bacteria of Peptococcus genera in the biological sample. According to yet another embodiment, the method comprises measuring the amount of bacteria of Peptostreptococcus genera in the biological sample. According to a further embodiment, the method comprises measuring the amount of bacteria of Parabacteroides genera in the biological sample. According to a further embodiment, the method comprises measuring the amount of Escherichia genera in the biological sample. According to a certain embodiment, the method comprises measuring the amount of bacteria of Verrucomicrobia in the biological sample. According to a further embodiment, the method comprises measuring the amount of bacteria of Lactobacillus genera in the biological sample. According to any one of the above embodiments, the method comprises measuring the amount of two said bacteria type, genera or species. According to some embodiments, the method comprises measuring the amount of three said bacteria types, genera or species. According to other embodiments, the method comprises measuring the amount of four said bacteria types, genera or species. According to yet another embodiment, the method comprises measuring the amount of five said bacteria types, genera or species. According to any one of the above embodiments, the sample is a sample of gut microbiome.

According to any one of the above embodiments, the method further comprises comparing the amount of said bacteria in said sample to an average amount of said bacteria in subjects having normal levels of active ADNP, wherein a significant difference between of the amount of said bacteria in comparison to the average amount in subjects having normal levels of active ADNP is predictive of ADNP deficiency.

The term “significant” has the meaning of statistical significance. In some embodiments, the difference is in 5, 10, 20, 30, 40, 50, 60, 70, 80, 90 or 100% in comparison to the bacterial load in subjects having normal levels of active ADNP. In some embodiments, the difference is by two-, three-, four-, five-, or ten-fold in comparison to the bacterial load in subjects having normal levels of active ADNP. Given individual variabilities, Log 10 calculations of population statistics might be used to obtain specific standards.

According to any one of the above embodiments, the subject is a human male subject. According to some embodiments, the at least one type of bacteria of gut microbiome is selected from Eubacteria, Lactobacillus group, Bifidobacterium, and Clostridium coccoides. According to one embodiment, the method comprises measuring the amount of bacteria of Eubacteria. According to another embodiment, the method comprises measuring and comparing the amount of bacteria of Bifidobacterium genus. According to yet another embodiment, the method comprises measuring and comparing the amount of bacteria of Lactobacillus group. According to some embodiments, the method comprises measuring and comparing the amount of Clostridium coccoides. According to any one of the above embodiment, the method comprises measuring and comparing two of said bacteria types, genera or species. According to some embodiments, the method comprises measuring and comparing three of said bacteria types, genera or species. According to other embodiments, the method comprises measuring and comparing four said bacteria types, genera or species. According to any one of the above embodiments, the sample is a sample of gut microbiome. The comparison is between the subject's sample and the average amount in subjects having normal levels of active ADNP.

The terms “normal bacterial load” and “normal amount of bacteria” are used herein interchangeably and refer to the average level of the bacteria in microbiome of subjects having normal levels of active ADNP.

According to some embodiments, a significant increase in the amount of the at least one type of the bacteria in comparison to the normal bacterial load of said type of is predictive of ADNP deficiency in the subject.

Thus, according to one embodiment, the present invention provides a method of detecting a male subject suffering from a ADNP deficiency, the method comprises: (i) measuring the amount of at least one type of bacteria selected from Eubacteria, Lactobacillus group, Bifidobacterium, and Clostridium coccoides in the gut microbiome of the subject; and (ii) comparing the amount determined in (i) to the normal amount of the bacteria, wherein if the amount measured in (i) is higher than the normal amount of said at least one type of bacteria, the subject is diagnosed as having ADNP deficiency.

According to one embodiment, the present invention provides a method of detecting a male subject suffering from a ADNP deficiency, the method comprises: (i) measuring the amount of Bifidobacterium in the gut microbiome of the subject; and (ii) comparing the amount determined in (i) to the normal amount Bifidobacterium, wherein if the amount measured in (i) is higher than the normal amount of said bacteria, the subject is diagnosed as having ADNP deficiency.

According to another embodiment, the present invention provides a method of detecting a male subject suffering from a ADNP deficiency, the method comprises: (i) measuring the amount of Eubacteria in the gut microbiome of the subject; and (ii) comparing the amount determined in (i) to the normal amount Eubacteria, wherein if the amount measured in (i) is higher than the normal amount of said bacteria, the subject is diagnosed as having ADNP deficiency.

According to yet another embodiment, the present invention provides a method of detecting a male subject suffering from a ADNP deficiency, the method comprises: (i) measuring the amount of bacteria of Lactobacillus group in the gut microbiome of the subject; and (ii) comparing the amount determined in (i) to the normal amount of bacteria of Lactobacillus group, wherein if the amount measured in (i) is higher than the normal amount of said bacteria, the subject is diagnosed as having ADNP deficiency.

According to a certain embodiment, the present invention provides a method of detecting a male subject suffering from a ADNP deficiency, the method comprises: (i) measuring the amount of Clostridium coccoides in the gut microbiome of the subject; and (ii) comparing the amount determined in (i) to the normal amount Clostridium coccoides, wherein if the amount measured in (i) is higher than the normal amount of said bacteria, the subject is diagnosed as having ADNP deficiency.

According to other embodiments, the subject is a female subject. According to some embodiments, the at least one type of bacteria of gut microbiome is selected from Enterococcus genus, Lactobacillus, Clostridium coccoides and Clostridium cluster IV.

According to one embodiment, the method comprises measuring and comparing the amount of bacteria of Enterococcus genus. According to yet another embodiment, the method comprises measuring and comparing the amount of bacteria of Lactobacillus group. According to some embodiments, the method comprises measuring and comparing the amount of Clostridium coccoides. According to some embodiments, the method comprises measuring and comparing the amount of Clostridium cluster IV. According to any one of the above embodiment, the method comprises measuring and comparing two of said bacteria types, genera or species. According to some embodiments, the method comprises measuring and comparing three of said bacteria types, genera or species. According to other embodiments, the method comprises measuring and comparing four said bacteria types, genera or species. The comparison is between the subject's sample and the average amount in subjects having normal levels of active ADNP (normal amount). According to any one of the above embodiments, the sample is a sample of gut microbiome.

According to one embodiment, the present invention provides a method of detecting a male subject suffering from a ADNP deficiency, the method comprises: (i) measuring the amount of Enterococcus genus in the gut microbiome of the subject; and (ii) comparing the amount determined in (i) to the normal amount Enterococcus genus, wherein if the amount measured in (i) is higher than the normal amount of said bacteria, the subject is diagnosed as having ADNP deficiency.

According to another embodiment, the present invention provides a method of detecting a male subject suffering from a ADNP deficiency, the method comprises: (i) measuring the amount of Clostridium coccoides in the gut microbiome of the subject; and (ii) comparing the amount determined in (i) to the normal amount Clostridium coccoides, wherein if the amount measured in (i) is higher than the normal amount of said bacteria, the subject is diagnosed as having ADNP deficiency.

According to yet another embodiment, the present invention provides a method of detecting a male subject suffering from a ADNP deficiency, the method comprises: (i) measuring the amount of bacteria of Lactobacillus group in the gut microbiome of the subject; and (ii) comparing the amount determined in (i) to the normal amount of bacteria of Lactobacillus group, wherein if the amount measured in (i) is lower than the normal amount of said bacteria, the subject is diagnosed as having ADNP deficiency.

According to yet another embodiment, the present invention provides a method of detecting a male subject suffering from a ADNP deficiency, the method comprises: (i) measuring the amount of bacteria of Clostridium cluster IV in the gut microbiome of the subject; and (ii) comparing the amount determined in (i) to the normal amount of bacteria of Clostridium cluster IV, wherein if the amount measured in (i) is lower than the normal amount of said bacteria, the subject is diagnosed as having ADNP deficiency.

According to any one of the above embodiments, the method, wherein the method further comprises providing recommendations to treat a disease related to ADNP-deficiency. According to one embodiment, the ADNP-deficiency related disease or condition is selected from Autism spectrum disorder (ASD), Alzheimer's disease (AD), Parkinson's disease (PD), schizophrenia, attention deficit disorder (ADHD), post-traumatic stress syndrome (PTSD), tauopathy, mild cognitive impairment, anxiety, depression, and muscle dysfunction. According to another embodiment, the ADNP-deficiency related disease is Autism spectrum disorder (ASD).

According to one embodiment, recommendations of treatment comprise recommendations to administer a pharmaceutical composition comprising an active agent selected from NAP peptide, SKIP peptide and ketamine to the subject. According to one embodiment, the method compares administering NAP peptide.

The term “administering” or “administration of” a substance, a compound or an agent to a subject can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2 times a day. According to other embodiments, the composition is administered 1, 2, 3, 4, 5 or 6 times a week or a month. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the administration is intranasal administration or subcutaneous administration.

According to some embodiments, the method of detecting of a subject suffering from a deficiency of activity-dependent neuroprotective protein (ADNP) comprises:
i. measuring the profile of the microbiome in a biological sample obtained from a subject;
ii. comparing the profile of the microbiome to the profile of a plurality of healthy subjects; and
iii. a significant difference between the profile of bacteria in (i) and (ii) indicates that the subject has the ADNP deficiency.

According to some embodiments, the microbiome is gut microbiome.

According to some embodiments, the method further comprises recommendation to monitor response to the treatment as described in any one of the above aspects and embodiments.

According to another embodiment, the sample is a saliva sample and the microbiome is a saliva microbiome. According to some embodiments, a significant difference between least one of the following types of bacteria and the normal level of said bacteria indicates that the subject is suffering from ADNP deficiency: Porphyromonas, Solobacterium, Haemophilus, Corynebacterium, Cellulosimicrobium, Streptococcus and Campylobacter, Porphyromonas gingivalis, Tannerella forsythia, Treponema denticola, Prevotella intermedia, Aggregatibacter actinomycemtomitans, Bacteroidetes, Firmicutes, Proteobacteria, Fusobacteria, Actinobacteria, Neisseria, Veilloneila and Fusobacterium. According to some embodiments, the ADNP-deficiency is a PTSD.

According to another aspect, the present invention provides a kit configured to monitor a response to a treatment of an ADNP-deficiency related disease or condition according to the methods as described in any one of the above embodiments and aspects. According to some embodiments, the present invention provides a kit for monitoring a response to a treatment of an ADNP-deficiency related disease or condition of an ADNP-deficient subject, the kit comprising: means for quantifying the amount of at least one type of bacteria in two or more samples of a microbiome of the subject at different time points and instructions to compare the amount of the at least one type of bacteria with the amount of said bacteria in a later sample from the subject, wherein the amount of said bacteria in the later measurement being closer to the normal amount of said bacteria is indicative of efficiency of the treatment. According to some embodiments, the means for quantifying the amount of at least one type of bacteria comprises means for DNA and/or RNA screening, amplification and quantification. Any methods known in the art may be used. The comparison to normal levels of the bacteria may be performed by comparison to known level as previously determined. According to some embodiments, the microbiome is a gut microbiome.

According to another aspect, the present invention provides a kit for determining a subject suffering from ADNP-deficiency to be used according to the methods as defined in any one of the above aspects and embodiments. According to one embodiment, the present invention provides a kit for determining a subject suffering from ADNP-deficiency, the kit comprises (i) means for quantifying the amount of at least one type of bacteria in a sample of a microbiome of the subject and (ii) means for comparing the amount obtained in (i) to the average amount of said at least one type of bacteria in subjects having normal levels of active ADNP, wherein a difference between the amount of (i) and the normal amount of the bacteria is indicative of ADNP-deficiency. According to some embodiments, the microbiome is gut microbiome.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### EXAMPLES

Materials and Methods

Animals

All procedures involving animals in the Adnp-mutated mouse model were approved by the Animal Care and Use Committee of Tel Aviv University and the Israeli Ministry of Health. Adnp heterozygous mice (Adnp^{+/-}) on an ICR background (an outbred mouse line), a model for the ADNP syndrome (Hacohen-Kleiman et al. 2018), were housed in a 12-h light/12-h dark cycle facility with free access to rodent chow and water. Genotyping was performed by Transnetyx (Memphis, Tenn.). After genotyping, the mice were housed in separate cages based on sex, genotype and treatment (up to five mice per cage), for two weeks prior to beginning of NAP treatment.

Peptide Synthesis and NAP Treatment

NAP peptide was custom made as described in (Hacohen-Kleiman et al. 2018). Prior to behavioral tests, intranasal treatment was administered daily to 1-month-old male and female mice (0.5 μg/5 μl/mouse/dose). For intranasal administration, the peptide was dissolved in a vehicle solution, termed DD, in which each milliliter included 7.5 mg of NaCl, 1.7 mg of citric acid monohydrate, 3 mg of disodium phosphate dihydrate, and 0.2 mg of benzalkonium chloride solution (50%). Each mouse was handheld in a semi-supine position with nostrils facing the investigator. A pipette tip was used to administer 5 μl/two nostrils. The mouse was handheld until the solution was entirely absorbed (˜10 s). Nasal NAP application was performed daily, once a day, for 45 days (5 days a week). After 45 days of treatment, in days of scheduled behavioral tests, NAP was applied 2 h before the test.

Based on previous studies both central peripheral effects were anticipated following intranasal administration, including previously observed effects on the immune system.

DNA Extraction Protocol for Molecular Murine Fecal Microbiota Analysis

Fecal pellets (1-2/mice) were collected 45 days after the beginning of treatment (NAP or DD) and stored at −80° C. until DNA purification procedures. The samples were thawed on ice mixed with 400 μl of sterile PBS/sample followed by mechanical disruption of the sample using a BeadBug microtube homogenizer (Daniel Biotech, Rehovot, Israel) for 2 minutes at 4,000 rpm. A 25 μl sample of lysozyme solution (20 mg/ml) was added, and the samples were incubated with shaking for 30 min at 37° C. on a thermo shaker racking platform. Next, 20 μl proteinase K (20 mg/ml) was included and the samples were thoroughly mixed with 400 μl of sterile lysis buffer (20 mM Tris HCl, 300 mM NaCl, 400 mM EDTA and 1% SDS) and incubated with shaking for 60 min at 56° C. on a thermo shaker racking platform. Next, 300 mg sterile zirconium beads and 150 μl of phenol was added to the samples followed by homogenization (twice for 40 sec. at 4,000 rpm, Daniel biotech, Rehovot, Israel). Then, 150 μl of CI-solution (chloroform-isoamyl alcohol 24:1) was added, mixed and submitted for centrifugation 13,000 rpm at room temperature for 5 minutes, Eppendorf Centrifuge 5417R (Eppendorf, Hamburg, Germany). A 600 μl volume of supernatant was removed and placed in a sterile 2 mL Eppendorf tube. 150 μl of CI-solution was then included and the samples were subjected to centrifugation as above. A 400 μl of aqueous, clear top phase was removed and placed in a sterile 2 mL Eppendorf tube. Then, the 400 μl of DNA solution was mixed with 100 μl of precipitation solution (40 mM EDTA 1.2M sodium acetate and 4 mg/ml glycogen). 1.3 mL of ice-cold 100% ethanol was added, the samples were then mixed and stored over night at −20° C. for DNA precipitation followed by centrifugation (13,000 rpm) at 4° C. The supernatant was discarded and the pellet washed with 500 μl ice-cold 70% ethanol followed by centrifugation for 5 minutes at 13,000 rpm, 4° C. (this step was repeated twice). The pellet was dried in the Speed Vac (Eppendorf Concentrator 5301) for 15 minutes and then re-suspended in 100 μl sterile water. Next, the samples were mixed on the shaker for 10 minutes at room temperature. In the next step, the samples were further purified using Qiagen Purification Kit according to the manufacturer's instructions (Thermo Fisher Scientific, Hilden, Germany). DNA was eluted in 100 μl sterile water and stored at −20° C. for long storage.

Murine Fecal Microbiota Analysis

In brief, DNA was further quantified by using Quant-iT PicoGreen reagent (Invitrogen, UK) and adjusted to 1 ng/μl. Then, the main bacterial groups abundant in the murine intestinal microbiota including Enterobacteria, Enterococci, Lactobacilli, Bifidobacteria, Bacteroides/Prevotella species, Clostridium coccoides group, Clostridium leptum group, Mouse intestinal Bacteroides (MIB) and total Eubacterial loads were determined by quantitative real-time polymerase chain reaction (qRT-PCR) with species-, genera-, or group-specific 16S rRNA gene primers (TibMolBiol, Germany) as reported previously, and numbers of 16S rRNA gene copies per nanogram DNA of each sample were assessed (as described in Escher et al., (Journal of microbiology & immunology 8:34-40 doi: 10.1556/1886.2018.00006).

Behavioral Analyses:

Open Field

The test provides a unique opportunity to systematically assess novel environment exploration, general locomotor activity, and anxiety-related behavior in rodents. Before executing any cognition assessing behavioral work, it is essential to recognize whether the animal behaves in a generally normal manner and to rule out abnormal physiology or motor problems such as ataxia etc. that would affect the proper course of the behavioral experiments. The open field apparatus is a 50×50 cm square arena, with 30 cm high walls, and all colored white. Mice were individually placed in the corner of the open field and left to explore freely for 15 min. The distance moved and time spent in the entire open field, as well as in its inner defined quadrants (center, border) was recorded using the EthoVision XT video tracking system and software (Noldus Inc. Leesburg, VA).

Social Approach and Social Memory

A plexiglas box is divided into three adjacent chambers, each 20 cm (length)×40.5 cm (width)×22 cm (height), separated by two removable doors. Steel wire pencil cups (10.16 cm (diameter), 10.8 cm (height)), are used as both containment for the target mice and as inanimate objects (weights prevent the mice from overturning the cups). Experiments were conducted on light during the dark phase of the mouse. Target mice (males for males and females for females) are placed inside the wire cup in one of the side chambers for three 10-min sessions on the day before the test for habituation. The next day, each subject mouse is tested in an experiment with three phases, each 10-min long (measured with a simple timer): I and II, the habituation phases (ensuring no bias), and III, the experimental phase. In phase III, an empty wire cup (novel object) is placed in the center of the right or left chamber and the cup containing the target mouse is placed in the center of the other chamber. Location of the empty wire cup (novel object) and the novel mice is counterbalanced to avoid confounding side preference. The doors are then removed and a 10-min timer is initiated. The three-chamber apparatus is cleaned between mice. The social approach task is also used as habituation for the social memory task, 3 h after the first phase (3-min exposure), the mouse is placed back into the apparatus for another 3 min (second phase), during which one cup contains the familiar mouse and the other contains a novel mouse. The positions of the familiar and novel mouse during phases 1 and 2 is counterbalanced within and between groups to exclude the possibility of positional effects but is kept the same for a given animal.

Mice were subjected to the tests as above. Mouse movement and exploratory behavior was tracked and recorded using the EthoVision XT video tracking system and software (Noldus Inc. Leesburg, Va.). The discrimination capacity (social memory) was analyzed using the formula: D2=(b−a)/(b+a).

Statistical Analysis

The effects of the ADNP-deficient genotype on microbiota composition in the Adnp^{+/-} mouse model were statistically tested by the Two-way ANOVA model with interaction. Both genotype and treatment (Adnp, NAP) were fitted as fixed-factors, for males and females separately. The data was analyzed after applying the logarithmic transformation since measurements showed skewness to the right. Statistical significance of the main effects of treatment, genotype, and their interaction was tested via F-test. Post-hoc analysis for pairwise comparisons across treatment\genotype were performed with Fisher's LSD for multiple comparisons. Two-way ANOVA analysis with Fisher's LSD as post hoc was utilized for behavioral analysis. Pearson's correlations were used for further comparisons. Further details are available in the figure and table legends.

### Example 1. Sex-Dependent Differences in Gut Microbiota Composition in Adnp Deficient Mice and the Effect of NAP Treatment

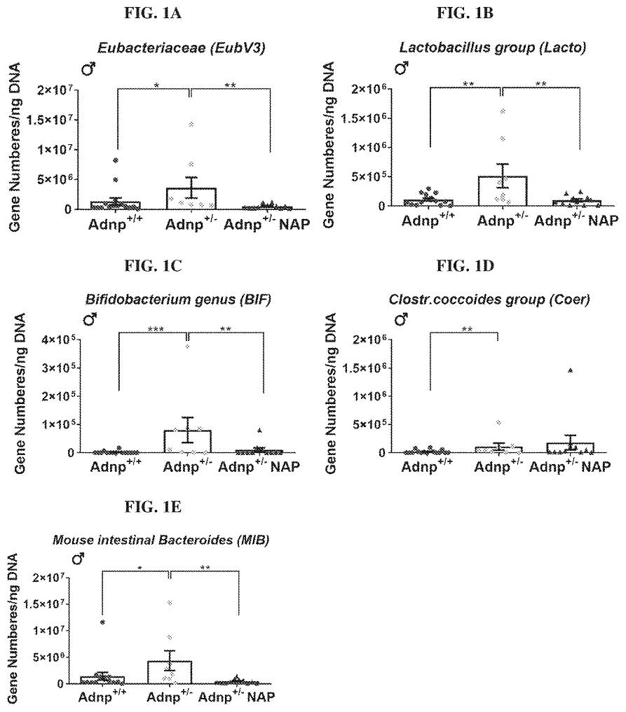

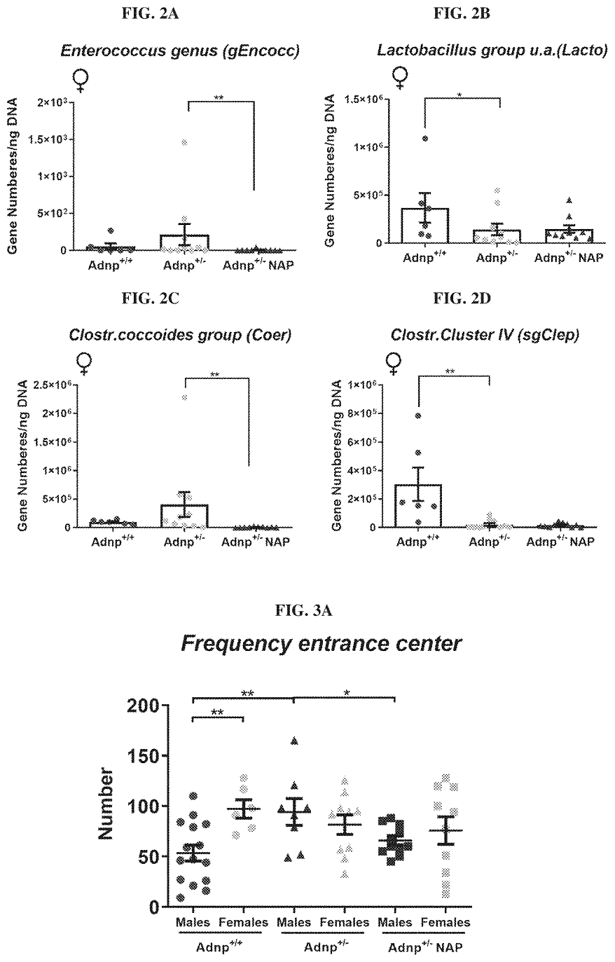

Several bacterial genera and species showed significant sex-dependent difference in abundance in Adnp deficient mice. As can be seen from FIG. 1, abundance of the following bacterial groups was significantly increased in Adnp-deficient male mice in comparison to Adnp+/+ mice Total Eubacterial loads (EubV3) (FIG. 1A), Lactobacillus group (Lacto) (FIG. 1B), Bifidobacterium genus (BIF) (FIG. 1C), Clostridium coccoides group (Coer) (FIG. 1D) and Mouse Intestinal Bacteroides (MIB) (FIG. 1E). All of the mentioned genera except for Clostridium coccoides group (Coer) decreased to control levels following NAP treatment. In female mice we observed the that the following genera/species significantly altered in Adnp deficient mice in comparison to Adnp^{+/+} mice or decreased to control levels following NAP treatment: Enterococcus genus (gEncocc) (FIG. 2A), Lactobacillus group (Lacto) (FIG. 2B) Clostridium coccoides group (Coer)(FIG. 2C) and Clostridium Cluster IV (sgClep) (FIG. 2D).

For the Clostridium coccoides group significant increases were observed in the male Adnp^{+/-} mice compared to male Adnp^{+/+} controls, whereas significant NAP-dependent decreases in Clostridium coccoides were observed in Adnp^{+/-} females. Decreased Lactobacillus species loads, however, were reveled in fecal samples derived from Andp^{+/-} females, contrasting the observed increases in respective species in male counterparts. Following NAP treatment, lower fecal Enterococcus genus (gEncocc) numbers were obtained in Adnp^{+/-} females only. Furthermore, in females, as opposed to male counterparts, Adnp deficiency resulted in decreasing intestinal load of the Clostridium leptum group (Cluster IV, sgClep) (FIG. 2D).

The bacterial groups that were neither affected by genotype nor by NAP treatment are provided below:

For females: Eubacteriaceae (EubV3); Bacteroides/Prevotella spp (Bac); Bifidobacterium genus (BIF); Mouse Intestinal Bacteroides (MIB); Enterobacteriaceae (Entero);

For males: Bacteroides/Prevotella spp (Bac); Enterococcus genus (gEncocc); Enterobacteriaceae (Entero); Clostridium Cluster IV (sgClep).

Additionally, of all the bacterial groups tested, NAP treatment resulted in essentially lower total Eubacterial loads in fecal samples derived from female Adnp^{+/+} mice, whereas higher intestinal numbers of Clostridium coccoides group (Coer) were observed in NAP treated compared to placebo treated male Adnp^{+/+} controls data not shown).

In summary, when comparing gut microbiota composition in the currently studied Adnp heterozygous mice (specifically, Adnp^{+/-} on an ICR background), we found dramatic differences between males and females. Thus, when assessing the main bacterial group abundance in the murine intestinal microbiota including Enterobacteria, Enterococci, Lactobacilli, Bifidobacteria, Bacteroides/Prevotella species, Clostridium coccoides group, Clostridium leptum group, Mouse intestinal Bacteroides (MIB) and total Eubacterial loads, essentially no overlap was observed for the Adnp^{+/-} genotype effect between males and females. While significant genotype effects were observed in five different microbiota groups in males, only two different microbiota groups were found to be significantly affected in females. Similarly, while NAP treatment reversed the male genotype deficits in four out of five genotype affected microbiota groups, in females it significantly corrected two trending genotype increases. Together the data suggests a more significant effect on males and an overall NAP corrective effect.

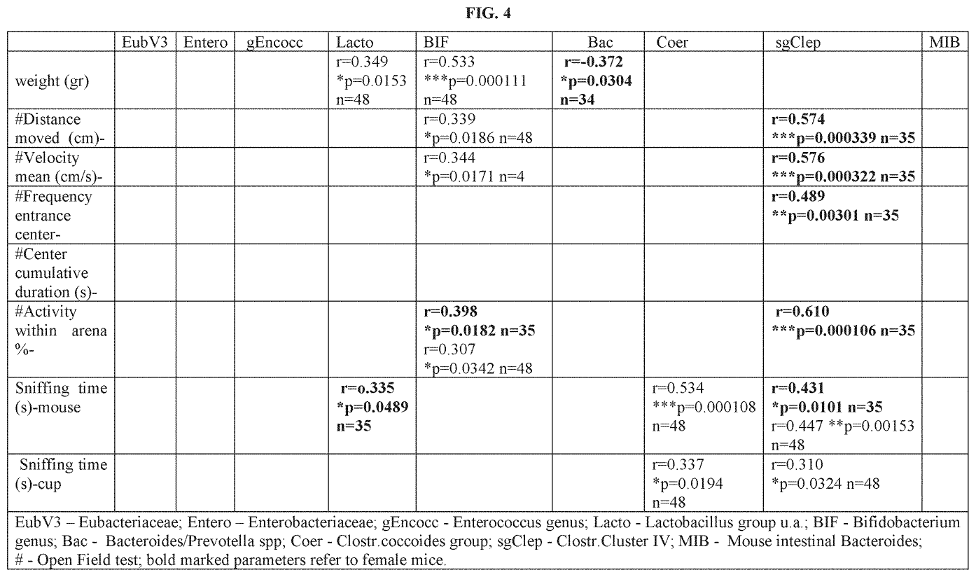

Additionally, the higher fecal Bacteroides/Prevotella numbers in Adnp^{+/-} females were shown to be associated with lower body weights (FIG. 4, r=−0.372 *p=0.0304, n=34). Whereas the Adnp^{+/-} female mice displayed lower body weights than female Adnp^{+/+} controls, NAP treatment resulted in increased weight in the male Adnp^{+/-} cohort.

Significant correlations were discovered among individual levels of weight, behaviors and specific bacterial loads (FIG. 4). In males, most measured parameters correlated with the fecal Bifidobacterium genus (BIF) load, while in females most measured parameters significantly correlated with intestinal Clostridium Cluster IV (sgClep) loads, with some significant behavior/bacterial loads correlations in both sexes including open field activity (Bifidobacterium genus (BIF) load), Clostridium Cluster IV (sgClep) (FIG. 4).

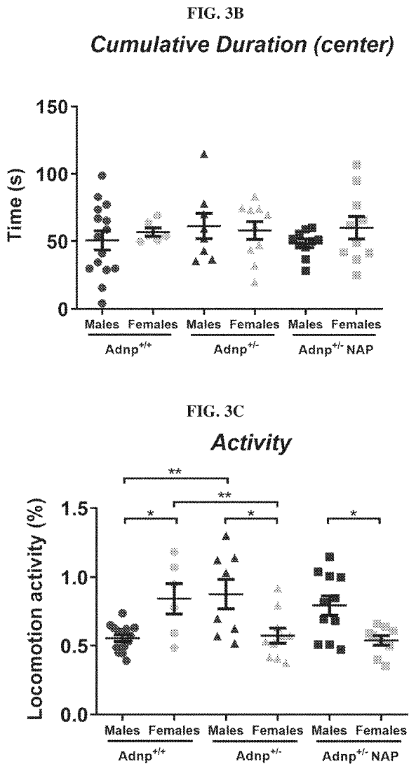

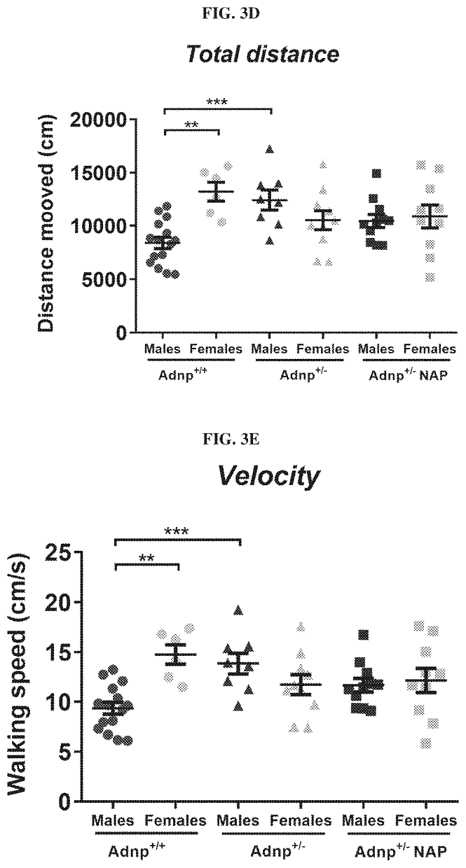

In summary, similar to the effects seen in gut microbiota composition, male behavioral parameters were found here to be more significantly affected compared to females as apparent in the open field (FIG. 3). This is further demonstrated in FIG. 4 showing sexual dichotomy in male—female behavior in correlation with gut bacterial group differences.

### DISCUSSION

In the present study we were able to reveal significant correlations of the gut microbiota composition of Adnp deficient mice and behavioral outcomes. We also showed that treatment by NAP peptide partially reverses both the behavioral changes as well as changes in microbiota. Therefore, changes in microbiota may be an important and significant indicator for ADNP deficiency for efficiency of treatment of ADNP-deficient subject, in particular treatment with NAP peptide.

### Example 2

We have developed another Adnp mutated mouse model using CRISP/Cas9 technology. The mice carry the most prevalent ADNP syndrome mutation (e.g. Adnp p.Tyr718* in the mouse corresponding to ADNP pTyr719* in the human). We have now discovered that these mice have aberrant visual evoked potential patterns (discovered in males), and this is rapidly corrected by NAP treatment. Without being limited to a particular theory it is suggested that this aberrant visual response is probably affected by visual cortex degeneration that is caused by Tau pathology, mimicking the situation in ADNP children. Monitoring visual evoked potential provides an objective observation of aberrant ADNP activity on one hand and its correction by treatment, e.g. by NAP peptide on another hand. Given the results in Example 1, gut microbiota samples are now being analyzed and are correlated also to visual evoked potential responses of males and females, coupling biomarkers for better diagnostics and clinical monitoring. The following bacteria in gut microbiota are tested and correlated with changes in visual evoked potential in subjects having aberrant ADNP activity: Bifidobacterium, Clostridium, Bacteroides, Faecalibacterium, Eubacterium, Ruminococcus, Peptococcus, Peptostreptococcus, Parabacteroides, Escherichia coli, Verrucomicrobia, Lactobacillus, Clostridium coccoides group, Mouse Intestinal Bacteroides (MIB) Enterococcus genus (gEncocc) and Clostridium Cluster IV (sgClep).

### Example 3

Human DNA, human fecal DNA, human saliva DNA and human sweat DNA from ADNP afflicted children and from their family members is collected and analyzed by the methodology described above. The results stratified with the general population. RNA sequencing (amplicon 16S) is performed by sequencing service. The microbiota signatures for male and female ADNP children is identified, compared with normal subjects and correlated with complete cytokine and chemokine profiles and immune status for diagnostic and monitoring purposes.

The following bacteria in gut microbiota are tested and correlated with complete cytokine and chemokine profiles in subjects having aberrant ADNP activity: Bifidobacterium, Clostridium, Bacteroides, Faecalibacterium, Eubacterium, Ruminococcus, Peptococcus, Peptostreptococcus, Parabacteroides, Escherichia coli, Verrucomicrobia, Lactobacillus, Clostridium coccoides group, Mouse Intestinal Bacteroides (MIB) Enterococcus genus (gEncocc) and Clostridium Cluster IV (sgClep).

The following bacteria in saliva microbiota are tested and correlated with complete cytokine and chemokine profiles in subjects having aberrant ADNP activity: Porphyromonas, Solobacterium, Haemophilus, Corynebacterium, Cellulosimicrobium, Streptococcus and Campylobacter, Porphyromonas gingivalis, Tannerella forsythia, Treponema denticola, Prevotella intermedia, Aggregatibacter actinomycemtomitans, Bacteroidetes, Firmicutes, Proteobacteria, Fusobacteria, Actinobacteria, Neisseria, Veillonella and Fusobacterium.

### Example 4

The goal of this experiment is to elucidate the correlation between saliva microbiota and susceptibility to post traumatic stress syndrome (PTSD) development. We examined and compared the microbiota composition in saliva of subjects suffering from PTSD and healthy people in order to elucidate parameters that may be used as a diagnostic measure for detecting subject susceptibility to PTSD and may be also used for monitoring their response to treatment. Saliva was collected from a cohort of PTSD veterans and appropriate controls. Prepared DNA is further subjected to 16S amplicon RNA sequencing. Microbiota composition then correlated with behavioral outcomes. Bacterial loads of the following bacteria are estimated: Porphyromonas, Solobacterium, Haemophilus, Corynebacterium, Cellulosimicrobium, Streptococcus and Campylobacter, Porphyromonas gingivalis, Tannerella forsythia, Treponema denticola, Prevotella intermedia, Aggregatibacter actinomycemtomitans, Bacteroidetes, Firmicutes, Proteobacteria, Fusobacteria, Actinobacteria, Neisseria, Veillonella and Fusobacterium. Alteration, i.e. increase or decrease in bacterial load of at least on type of the above bacteria is correlated with increased susceptibility to PTSD, whereas treatment of PTSD is correlated to reverse of said bacterial to normal bacterial load.

Although the present invention has been described herein above by way of preferred embodiments thereof, it can be modified, without departing from the spirit and nature of the subject invention as defined in the appended claims

1.-37. (canceled)

38. A method for monitoring a response to a treatment of disease or condition related to deficiency of activity-dependent neuroprotective protein (ADNP) in an ADNP-deficient subject comprising measuring the amount of at least one type of bacteria of a microbiome in a biological sample obtained from the subject at two or more different time points, wherein the patient responds favorably to the treatment if the amount of said bacteria in the later measurement changes in amount to a value which is closer to the average amount of said bacteria in subjects having normal levels of active ADNP than in the previous measurement.

39. The method according to claim 38, characterized by at least one of:
(i) the biological sample is selected from fecal, saliva and sweat sample;
(ii) measuring the amount of at the at least one type of bacteria comprises RNA 16S analysis of the microbiome;
(iii) the biological sample is a fecal sample and the microbiome is gut microbiome;
(iv) the biological sample is a fecal sample and the at least one type of bacteria is selected from Bifidobacterium, Clostridium, Bacteroides, Faecalibacterium, Eubacterium, Ruminococcus, Peptococcus, Peptostreptococcus, Parabacteroides, Escherichia coli, Verrucomicrobia and Lactobacillus.

40. The method according to claim 38, wherein the biological sample is a fecal sample and the subject is a male subject.

41. The method according to claim 40, characterized by at least one of (i) the at least one type of bacteria is selected from Eubacteria, Lactobacillus group, Bifidobacterium, and Clostridium coccoides; and (ii) the at least one type of bacteria is of Bifidobacteria genus.

42. The method according to claim 41, wherein a decrease in the amount of the at least one type of bacteria between the previous and the later measurements is indicative of favorable response of the subject to treatment.

43. The method according to claim 38, wherein the biological sample is a fecal sample and the subject is a female.

44. The method according to claim 43, wherein the at least one type of bacteria is selected from Enterococcus genus, Lactobacillus, Clostridium coccoides and Clostridium cluster.

45. The method according to claim 44, wherein (i) a decrease in the amount of at least one type of bacteria selected from Enterococcus and Clostridium coccoides between the previous and the later measurements; (ii) an increase in the amount of at least one type of bacteria selected from Lactobacillus and Clostridium cluster IV between the previous and the later measurements; or both (i) and (ii) is indicative of favorable response of the subject to treatment.

46. The method according to claim 38, characterized by at least one of:
(i) the treatment comprises administering a pharmaceutical composition comprising an active agent selected from NAP peptide, SKIP peptide, and ketamine;
(ii) the ADNP-deficiency comprises reduced levels of active ADNP and mutation reducing activity of ADNP protein;
(iii) the ADNP-deficiency related disease or condition is selected from Autism spectrum disorder (ASD), Alzheimer's disease (AD), Parkinson's disease (PD), schizophrenia, attention deficit disorder (ADHD), post-traumatic stress syndrome (PTSD), taupathy, mild cognitive impairment, anxiety, depression, and muscle dysfunction; and
(iv) at least one of the measurements is performed before the beginning of the treatment.

47. A method of detecting a subject suffering from a deficiency of activity-dependent neuroprotective protein (ADNP), the method comprising:
(i) measuring the amount of at least one type of bacteria in the microbiome of the subject; and
(ii) comparing the amount determined in (i) to the average amount of the bacteria in subjects having normal ADNP levels,
wherein a significant difference between of the amount of said bacteria measured in (i) in comparison to the average amount in subjects having normal levels of active ADNP is predictive of ADNP deficiency.

48. The method according to claim 47, characterized by at least one of:
(i) the biological sample is selected from a fecal, saliva sample and sweat sample;
(ii) measuring the amount of at the at least one type of bacteria comprises RNA 16S analysis of the microbiome;
(iii) the biological sample is a fecal sample and the microbiome is a gut microbiome;
(iv) the biological sample is a fecal sample and the at least one type of bacteria is selected from Bifidobacterium, Clostridium, Bacteroides, Faecalibacterium, Eubacterium, Ruminococcus, Peptococcus, Peptostreptococcus, Parabacteroides, Escherichia coli, Verrucomicrobia and Lactobacillus.

49. The method according to claim 47, wherein the biological sample is a fecal sample, the subject is a male subject and at least one type of bacteria is bacteria selected from Eubacteria, Lactobacillus group, Bifidobacterium, Clostridium coccoides and Bifidobacteria genus.

50. The method according to claim 49, wherein a significant increase in the amount of the at least one type of the bacteria in comparison to the average amount of the type of bacteria in subjects having normal ADNP levels is predictive of ADNP deficiency in the subject.

51. The method according to claim 47, wherein the biological sample is a fecal sample, the subject is a female subject and the at least one type of bacteria is selected from Enterococcus genus, Lactobacillus, Clostridium coccoides and Clostridium cluster IV.

52. The method according to claim 51, wherein (i) a significant increase in the amount of bacteria selected from Enterococcus and Clostridium coccoides in comparison to the average amount of the bacteria in subjects having normal ADNP levels; (ii) a significant decrease in the amount of bacteria selected from Lactobacillus and Clostridium cluster IV in comparison to the average amount of the bacteria in subjects having normal ADNP levels; or (iii) both (i) and (ii) is predictive of ADNP deficiency in the subject.

53. The method according to claim 47, wherein the method further comprises providing recommendations to treat a disease or condition related to ADNP-deficiency.

54. The method according to claim 53, characterized by at least one of (i) the ADNP-deficiency related disease or condition is selected from Autism spectrum disorder (ASD), Alzheimer's disease (AD), Parkinson's disease (PD), schizophrenia, attention deficit disorder (ADHD), post-traumatic stress syndrome (PTSD), taupathy, mild cognitive impairment, anxiety, depression, and muscle dysfunction; and (ii) the treatment comprises administering a pharmaceutical composition comprising an active agent selected from NAP peptide, SKIP peptide and ketamine to the subject.

55. The method according claim 53, further consulting monitoring a response to the treatment by the method of claim 38.

56. A kit for monitoring a response to a treatment of an ADNP-deficiency related disease or condition of an ADNP-deficient subject, the kit comprising: means for quantifying the amount of at least one type of bacteria in two or more samples of a microbiome of the subject at different time points and instructions to compare the amount of the at least one type of bacteria with the amount of said bacteria in a two samples from the subject, wherein the amount of said bacteria in the later measurement being closer to the average amount of said bacteria in subjects having normal levels of active ADNP is indicative of efficiency of the treatment, optionally wherein the microbiome is a gut microbiome.

57. A kit for determining a subject suffering from ADNP-deficiency, the kit comprises (i) means for quantifying the amount of at least one type of bacteria in a sample of a microbiome of the subject and (ii) means for comparing the amount obtained in (i) to the average amount of said at least one type of bacteria in subjects having normal levels of active ADNP, wherein a difference between the amount of (i) and the average amount of said type of bacteria in subjects having normal levels of active ADNP is indicative of ADNP-deficiency, optionally wherein the microbiome is a gut microbiome.
